(19) United States
(12) Patent Application Publication
(10) Pub. No.: US 2020/0246396 A1
(43) Pub. Date: Aug. 6, 2020

(54) INHIBITION OF COLONIC GROUP 3 INNATE LYMPHOID CELLS

(71) Applicants: President and Fellows of Harvard College, Cambridge, MA (US); Children's Medical Center Corporation, Boston, MA (US)

(72) Inventors: Neeraj K. Surana, Boston, MA (US); Dennis Kasper, Brookline, MA (US)

(21) Appl. No.: 16/312,815

(22) PCT Filed: Jun. 22, 2017
(86) PCT No.: PCT/US2017/038680

§ 371 Date: Dec. 21, 2018

Related U.S. Application Data
(60) Provisional application No. 62/353,384, filed on Jun. 22, 2016.

(51) Int. Cl. A61K 35/741; A23L 33/135; A61P 1/00

(52) U.S. Cl. CPC A61K 35/741; A61P 1/00; A23L 33/135

(57) ABSTRACT

In certain aspects, described herein are methods, bacteria, and compositions for the reduction of the amount, activity and/or proliferation of colonic group 3 innate lymphoid cells (ILC3s) and for the treatment and/or prevention of diseases associated with pathological immune responses, such as inflammatory bowel diseases.

### RELATED APPLICATIONS

This application is a § 371 national-stage application based on PCT/US17/38680 filed Jun. 22, 2017 which claims the benefit of priority to U.S. Provisional Application No. 62/353,384 filed on Jun. 22, 2016, which is hereby incorporated in its entirety.

### GOVERNMENT INTEREST

This invention was made with government support under Grant Number AI108690, awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 23, 2017, is named HMV-26625_SL.txt and is 7,080,032 bytes in size.

### BACKGROUND

Inflammatory bowel disease (IBD) is a group of inflammatory conditions of the colon and small intestine. The two most common forms of IBD, Crohn's disease and ulcerative colitis, affect over 2.5 million people of European ancestry, with rising prevalence in other populations. Treatments for IBD include diet and lifestyle changes, drug therapy, and surgery. Currently, there is no cure for IBD. Instead, the goal of treatment focuses on attaining remission. Thus, there is a great need for improved compositions and methods for the treatment of IBD and other autoimmune and inflammatory conditions.

### SUMMARY

In certain aspects, provided herein is a method of reducing the amount, activity (e.g., IL-17 secretion) and/or proliferation of colonic group 3 innate lymphoid cells (ILC3s) in a subject. In certain embodiments, the method comprises administering to the subject a composition comprising a bacteria of the family Lachnospiraceae. In some embodiments, the subject suffers from or is predisposed to an immune disorder, such as an inflammatory bowel disease, psoriasis, or asthma. In some embodiments, the immune disorder is an inflammatory bowel disease, such as Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease. In some embodiments, the inflammatory bowel disease is ulcerative colitis. In some embodiments of the methods described herein, the administration of the composition reduces the number of IL-17 expressing colonic ILC3s in the subject.

In some embodiments of the methods described herein, the composition administered to the subject further comprises a pharmaceutically acceptable carrier. In some embodiments, the composition is administered orally. In some embodiments of the methods and compositions described herein, the composition is a food product supplemented with the bacteria. In some embodiments, the composition is administered rectally.

In some embodiments, the methods described herein further comprise administering a pre-biotic and/or administering an antibiotic. In some embodiments, the method comprises administering an antibiotic to the subject prior to the administration of the composition.

In certain aspects, provided herein is a pharmaceutical composition comprising a bacteria of the family Lachnospiraceae and a pharmaceutically acceptable carrier. In some embodiments, the composition is formulated for oral administration. In some embodiments, the composition is formulated for rectal administration. In some embodiments, the composition further comprises a pre-biotic. In some embodiments of the methods and compositions described herein, the composition is a food product supplemented with the bacteria.

In some embodiments of the methods and compositions described herein, the bacteria of the family Lachnospiraceae are of the genus Acetitomaculum, Anaerofilum, Anaerostipes, Blautia, Butyrivibrio, Catenibacterium, Catonella, Clostridium, Coprococcus, Dorea, Epulopiscium, Johnsonella, Lachnobacterium, Lachnospira, Oribacterium, Pseudobutyrivibrio, Roseburia, Ruminococcus, Shuttleworthia or Sporobacterium. In some embodiments of the methods and compositions described herein, at least 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the bacteria in the composition are of the family Lachnospiraceae. In some embodiments of the methods and compositions described herein, the bacteria are live, replication competent bacteria.

In some aspects provided herein are bacteria and compositions (e.g., pharmaceutical compositions) comprising said bacteria, wherein the bacteria's 16s DNA has a sequence that is at least 95%, at least 99%, at least 99.5%, or at least 99.9% identical to SEQ ID NO: 1. In some embodiments, the bacteria's 16s DNA has a sequence that is identical to SEQ ID NO: 1. In some aspects, provided herein are bacteria having the genome comprising a sequence that is at least 90%, at least 95%, at least 99%, at least 99.5%, or at least 99% identical to SEQ ID NO: 2. In some embodiments, the genome sequence is identical to SEQ ID NO: 2. The bacteria (i.e., bacteria comprising 16s DNA having a sequence that is at least partially identical to SEQ ID NO:1 and/or having genome sequence that is at least partially identical to SEQ ID NO: 2) may comprise at least 10%, 15%, 20%, 25%, 30%, 35%, 40%, 45%, 50%, 55%, 60%, 65%, 70%, 75%, 80%, 85%, 90%, 95%, 96%, 97%, 98%, or 99% of the bacteria in the composition. In some embodiments, the bacteria have undergone directed evolution. In some embodiments, the bacteria are not naturally occurring. In some embodiments, the composition formulated for oral administration (e.g., the composition may be a food product). In some embodiments, the composition is formulated for rectal administration. In some embodiments, the bacteria are live, replication competent bacteria. The composition may comprise a pre-biotic or an antibiotic.

Provided herein are methods of treating and/or preventing a condition (e.g., an immune disorder, such as an inflammatory bowel disease, psoriasis, or asthma) by administering bacteria or compositions comprising said bacteria to the subject. In some embodiments, the condition is an inflammatory bowel disease, such as Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease. In some embodiments, the composition reduces the amount, activity and/or proliferation of colonic group 3 innate lymphoid cells (ILC3s) in a subject.

Provided herein are compositions and methods related to reducing the amount, activity and/or proliferation of colonic group 3 innate lymphoid cells (ILC3s) in a subject by administering to the subject bacteria disclosed herein or a composition comprising said bacteria. In some embodiments, the subject suffers from or is predisposed to an inflammatory bowel disease (e.g., Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease), psoriasis, and/or asthma.

The bacteria and/or compositions disclosed herein may be administered orally or rectally. In some embodiments, the methods disclosed herein further comprise administering a pre-biotic or an antibiotic to the subject. The antibiotic or pre-biotic may be administered prior and/or after administration of the bacterium or composition.

Provided herein are methods of identifying bacteria that convey disease resistance and/or disease susceptibility by determining a first microbiome profile for a disease-susceptible subject and/or a disease-resistant subject, co-housing the disease-susceptible subject with the disease-resistant subject for a period of time sufficient for the disease-susceptible subject to acquire disease resistance, and, after co-housing, determining a second microbiome profile for the disease-susceptible subject and/or a disease-resistant subject. In some embodiments, the method comprises comparing the first microbiome profile of the disease-susceptible subject with the second microbiome profile of the disease-susceptible subject to identify operational taxonomic units (OTUs) of bacteria that are more prevalent in the second microbiome profile of the disease-susceptible subject than the first microbiome profile of the disease-susceptible subject. Operational taxonomic units (OTUs) of bacteria that are more prevalent in the second microbiome profile of the disease-susceptible subject than the first microbiome profile may confer disease-resistance. In some embodiments, the method comprises comparing the first microbiome profile of the disease-susceptible subject with the second microbiome profile to identify operational taxonomic units (OTUs) of bacteria that are less prevalent in the second microbiome profile of the disease-susceptible subject than the first microbiome profile of the disease-susceptible subject. Operational taxonomic units (OTUs) of bacteria that are less prevalent in the second microbiome profile of the disease-susceptible subject than the first microbiome of the disease-susceptible subject profile may confer disease susceptibility.

In some embodiments, the method comprises comparing the first microbiome profile of the disease-resistant subject with the second microbiome profile of the disease-resistant subject to identify operational taxonomic units (OTUs) of bacteria that are more prevalent in the second microbiome profile of the disease-resistant subject than the first microbiome profile of the disease-resistant subject. Operational taxonomic units (OTUs) of bacteria that are more prevalent in the second microbiome profile of the disease-resistant subject than the first microbiome profile of the disease-resistant subject may confer disease susceptibility. In some embodiments, the method comprises comparing the first microbiome profile of the disease-resistant subject with the second microbiome profile of the disease-resistant subject to identify operational taxonomic units (OTUs) of bacteria that are less prevalent in the second microbiome profile of the disease-resistant subject than the first microbiome profile of the disease-resistant subject. Operational taxonomic units (OTUs) of bacteria that are less prevalent in the second microbiome profile of the disease-resistant subject than the first microbiome of the disease-resistant subject profile may confer disease resistance.

In some aspects, provided herein are methods of identifying bacteria that convey disease-susceptibility and/or disease-resistance by determining a first microbiome profile for a disease-resistant subject, co-housing the disease-resistant subject with a disease-susceptible subject for a period of time sufficient for the disease-resistant subject to acquire disease susceptibility, and, after co-housing, determining a second microbiome profile for the disease-resistant subject. In some embodiments, the method comprises comparing the first microbiome profile of the disease-resistant subject with the second microbiome profile of the disease-resistant subject to identify operational taxonomic units (OTUs) of bacteria that are more prevalent in the second microbiome profile of the disease-resistant subject than the first microbiome profile of the disease-resistant subject. Operational taxonomic units (OTUs) of bacteria that are more prevalent in the second microbiome profile for the disease-resistant subject than the first microbiome profile of the disease-resistant subject may confer disease susceptibility. In some embodiments, the method comprises comparing the first microbiome profile of the disease-resistant subject with the second microbiome profile of the disease-resistant subject to identify operational taxonomic units (OTUs) of bacteria that are less prevalent in the second microbiome profile of the disease-resistant subject than the first microbiome profile of the disease-resistant subject. Operational taxonomic units (OTUs) of bacteria that are less prevalent in the second microbiome profile for the disease-resistant subject than the first microbiome profile for the disease-resistant subject may confer disease resistance.

In some embodiments, the method comprises comparing the first microbiome profile of the disease-susceptible subject with the second microbiome profile of the disease-susceptible subject to identify operational taxonomic units (OTUs) of bacteria that are more prevalent in the second microbiome profile of the disease-susceptible subject than the first microbiome profile of the disease-susceptible subject. Operational taxonomic units (OTUs) of bacteria that are more prevalent in the second microbiome profile of the disease-susceptible subject than the first microbiome profile of the disease-susceptible subject may confer disease resistance. In some embodiments, the method comprises comparing the first microbiome profile of the disease-susceptible subject with the second microbiome profile to identify operational taxonomic units (OTUs) of bacteria that are less prevalent in the second microbiome profile of the disease-susceptible subject than the first microbiome profile of the disease-susceptible subject. Operational taxonomic units (OTUs) of bacteria that are less prevalent in the second microbiome profile of the disease-susceptible subject than the first microbiome of the disease-susceptible subject profile may confer disease susceptibility. In some embodiments, the disease is inflammatory bowel disease (e.g., Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease), psoriasis, or asthma.

The subject may be a human or a non-human animal, such as a rodent (e.g., mouse or rat). In some embodiments, at least a portion of the subject's microbiome comprises bacteria from another species. For example, the subject may be a mouse, and have a microbiome comprising bacteria naturally occurring in humans.

The period of time may be any time needed to exchange microbiome bacteria between subjects. In some embodiments, the period of time is any time needed to confer disease resistance or disease susceptibility. For example, the time may be 12 hours, 24 hours, 36 hours, 48 hours, 3 days, 5 days, 1 week, 2 weeks, 3 weeks, 6 weeks, or 12 weeks.

In some embodiments, the compositions described herein (i.e., compositions comprising a bacterium disclosed herein) further comprise additional bacteria (e.g. bacteria of a different species or genus). The additional bacteria may be of the family Lachnospiraceae. In some embodiments, the additional bacteria of the family Lachnospiraceae are of the genus Acetitomaculum, Anaerofilum, Anaerostipes, Blautia, Butyrivibrio, Catenibacterium, Catonella, Clostridium, Coprococcus, Dorea, Epulopiscium, Johnsonella, Lachnobacterium, Lachnospira, Oribacterium, Pseudobutyrivibrio, Roseburia, Ruminococcus, Shuttleworthia or Sporobacterium.

### BRIEF DESCRIPTION OF THE DRAWINGS

FIG. 1 is a mouse survival curve showing that mice having normal mouse microbiota (MMb) are sensitive to dextan sodium sulfate (DSS) colitis, while mice having normal human microbiota (HMb) are resistant to DSS colitis. SPF: Specific Pathogen Free; GF: Germ Free.

FIG. 2 includes two panels (A and B) and shows the prevalence of several major colonic lymphoid (panel A) and myeloid (panel B) cell subsets in MMb and HMb mice.

FIG. 3 shows the number of colonic NKp46+ group 3 innate lymphoid cells (ILC3s) in MMb and HMb mice.

FIG. 4 depicts a schematic of the exemplary systems-based approach disclosed herein to identify immunomodulatory bacteria.

FIG. 5 includes two panels (A and B), each of which depict survival curves showing that the relationship between the duration of co-housing and DSS susceptibility. X:Y denotes that mouse type y was co-housed with mouse type x.

FIG. 6 depicts a principle coordinate analysis of 16S rDNA sequences from the feces of MMb, specific pathogen-free (SPF), and HMb mice.

FIG. 7 depicts the principle coordinate analysis of 16S rDNA sequences from the feces MMb, SPF, and HMb mice (as shown in FIG. 6), as well as HMb and MMb mice that were co-housed with each other for 1 day and then singly housed for another 13 days (d14). X:Y denotes that mouse type y was co-housed with mouse type x.

FIG. 8 is a table listing the total number of different operational taxonomic units (OTUs) present in each pairwise comparison of mouse types, and the number of differentially abundant taxa (identified by LEfSe) for each comparison.

FIG. 9 shows the relative abundance of Lachnospiraceae in the indicated mouse types.

FIG. 10 shows that culturing feces from HMb mice on semi-selective media resulted in a ˜5-fold enrichment of Lachnospiraceae.

FIG. 11 shows the number of NKp46+ ILC3s in MMb mice given the cultivable fraction from either MMb or HMb feces.

FIG. 12 includes two panels (A and B) showing the number of IL-22+(Panel A) and IL-17+(Panel B) NKp46+ ILC3s in MMb and HMb mice.

FIG. 13 is a survival curve showing that MMb mice given the cultivable fraction from HMb feces (circle) have less DSS-induced mortality then MMb mice administered the cultivatable fraction from MMb feces (square).

FIG. 14 shows MMb mice have more severe colitis than HMb mice. Weight change and survival of GF (red, square, n=12), MMb (n=10), HMb (green upside-down triangle; n=12), and SPF (n=12) mice subjected to DSS-induced colitis. Mean±SEM is depicted. Histologic assessment of disease severity in the colon at days 5 and 10 after induction of colitis in MMb, HMb, and SPF mice. Bars depict the mean+SEM.

FIG. 15 shows microbial pedigree analysis reveals that the bacterial family Lachnospiraceae is associated with survival from colitis. Survival of mice following DSS-induced colitis. A. MMb (blue; n=4) and HMb (green; n=4) mice were co-housed for 3 weeks before induction of colitis. Dashed and solid lines depict mice that were and were not co-housed, respectively. B. MMb mice were co-housed with HMb mice for 1 or 3 days (n=8 per group). C. HMb mice were co-housed with MMb mice for 1 (blue) or 3 days (red). Non-co-housed HMb mice are depicted in green (n=8 per group). D. A principal-components analysis depicting the fecal microbiota of MMb, SPF, HMb, MMb^{HMb-1d}, and HMb^{MMb-1d} mice. E. The different pairwise comparisons used as “family pedigrees,” with 4 mice included per group; the total number of operational taxonomic units (OTUs) in all 8 mice per pair; and the differentially abundant taxa, which were identified with LEfSe. The only taxon that was identified in all 4 comparisons was the family Lachnospiraceae. F. The relative abundance of Lachnospiraceae in each of the indicated microbiotas is shown.

FIG. 16. Clostridium immunis protects MMb mice from colitis. A. Relative abundance of bacterial families present in HMb feces before and after culture. B. Survival of MMb mice orally receiving MMb cx (blue, square; n=9) or HMb cx (green, triangle; n=9) and subjected to DSS-induced colitis. Data are pooled from 2 independent experiments. C. Relative abundance of Lachnospiraceae in a subset of the mice depicted in panel B. The fecal microbiota was assessed before gavage with any material (MMb; n=9) or 1 week after gavage with MMb cx (n=5) or HMb cx (n=4). The post-gavage samples represent day 0 of the DSS colitis experiment. The bars represent the mean+SEM. D. Survival of MMb mice orally receiving Clostridium innocuum (green, triangle; n=5) or Clostridium immunis (blue, square; n=5) and subjected to DSS-induced colitis.

FIG. 17 shows individual microbiome-wide association studies reveal a large number of differentially abundant taxa. LEfSe was used to identify differentially abundant taxa in the fecal microbiota of various mice. The red and green colors reflect taxa that are more abundant in that particular group. Taxa that are colored yellow did not statistically differ in abundance between groups. Each ring of the cladogram represents a different taxonomic level, starting with kingdom in the center and ending with genus in the outer ring. A. Comparison of HMb and MMb. B. Comparison of MMb and SPF. C. Comparison of MMb and MMb^{HMb-1d}. D. Comparison of HMb and HMb^{MMb-1d}. The family Lachnospiraceae is indicated by the symbols c4 (A), a6 (B), al (C), and a9 (D).

FIG. 18. shows that culture of MMb feces on semi-selective medium does not enrich for Lachnospiraceae. The relative abundance of bacterial families present in MMb feces before and after culture is shown.

FIG. 19 shows MMb mice given MMb cx or HMb cx have distinct microbiotas. Principal-components analysis of the fecal microbiota of MMb mice before and after gavage with MMb cx or HMb cx is shown. The arrow indicates an MMb mouse that received HMb cx but died after being challenged with DSS.

FIG. 20 shows HMb cx bacterial consortium is sufficient to protect mice from colitis-associated death. The survival of GF mice (n=10) orally receiving HMb cx and subjected to DSS-induced colitis is shown.

FIG. 21 shows FAME analysis demonstrates that C. immunis is different than the type strain of C. symbiosum. GC analysis of fatty acid methyl esters (FAME analysis) was performed by MIDI Labs. The identification based on this analysis was Clostridium symbiosum group B, but the type strain of C. symbiosum is in group A. Given this difference, it is clear that the newly identified species is distinct from C. symbiosum. The fact that it is named group A vs B reflects historical artifact as opposed to scientific similarity.

FIG. 22 shows C. immunis maintains disease remission in mice with microbiotas different than MMb. The left panel demonstrates that RMb mice (gnotobiotic mice harboring a rat microbiota) are very susceptible to DSS colitis. In the right panel, treatment of RMb mice with C. immunis one-week prior to challenge with DSS leads to improved survival.

FIG. 23 shows that C. immunis can treat pre-existing inflammation, MMb mice were subjected to DSS colitis. Animals were treated with either C. innocuum (control) or C. immunis on day 5, when significant colonic inflammation already exists. C. immunis-treated mice have less weight loss and improved survival compared to the C. innocuum-treated control animals.

FIG. 24 shows exemplary 16rs sequence of a non-naturally occurring bacterium.

### DETAILED DESCRIPTION

### General

In certain aspects, provided herein are bacterium (e.g., a non-naturally occurring bacterium), methods and compositions related to the reduction of the amount, activity (e.g., IL-17 production) and/or proliferation of colonic group 3 innate lymphoid cells (ILC3s) in a subject and/or for the treatment and/or prevention of diseases associated with pathological immune responses (i.e., an immune disorder), such as inflammatory bowel diseases, through the administration of a bacteria disclosed herein or a composition comprising the bacteria (e.g., a naturally occurring and/or non-naturally occurring bacterium) of the family Lachnospiraceae. In certain aspects, provided herein are bacteria (e.g. non-naturally occurring bacterium), compositions comprising said bacteria, and methods of treating and or preventing a condition (e.g., a condition disclosed herein) by administering to the subject the bacteria or a composition comprising the bacteria.

### Definitions

For convenience, certain terms employed in the specification, examples, and appended claims are collected here.

The articles “a” and “an” are used herein to refer to one or to more than one (i.e., to at least one) of the grammatical object of the article.

As used herein, the term “administering” means providing an agent or composition to a subject, and includes, but is not limited to, administering by a medical professional and self-administering.

As used herein, an “effective amount” is an amount effective in treating or preventing a disease associated with a pathological immune response, including, for example, inflammatory bowel disease.

As used herein, the term “immune disorder” refers to any disease, disorder or disease symptom caused by an activity of the immune system, including autoimmune diseases, inflammatory diseases and allergies.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

As used herein, the term “subject” means a human or non-human animal selected for treatment or therapy. In certain embodiments, of the methods and compositions described herein the subject is a human subject.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

Bacteria that Reduce Production of ILC3s

In certain aspects, provided herein are compositions and methods related the use of bacteria that reduce the production of colonic group 3 innate lymphoid cells (ILC3s). In certain embodiments, the bacteria are of the family Lachnospiraceae. In some embodiments, the bacteria are of the genus Acetitomaculum, Anaerofilum, Anaerostipes, Blautia, Butyrivibrio, Catenibacterium, Catonella, Clostridium, Coprococcus, Dorea, Epulopiscium, Johnsonella, Lachnobacterium, Lachnospira, Oribacterium, Pseudobutyrivibrio, Roseburia, Ruminococcus, Shuttleworthia or Sporobacterium. In some embodiments, the bacteria are a non-naturally occurring bacteria. In some embodiments, the bacteria are the product of directed evolution.

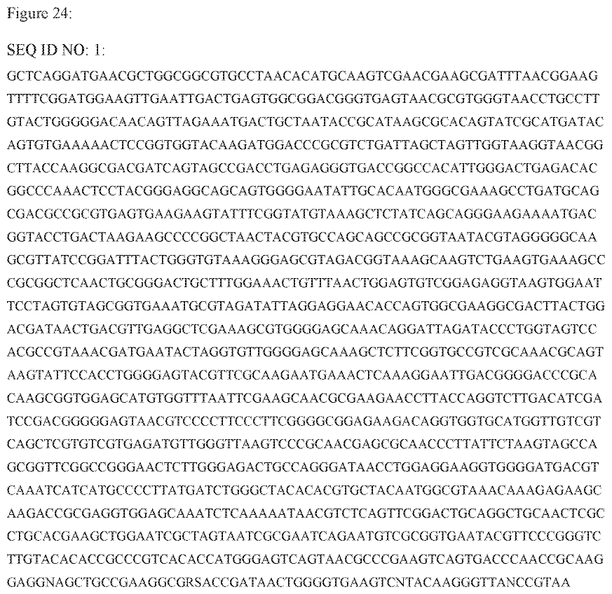

In some embodiments, the bacteria comprise DNA having a sequence that is at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, 99.9% or 100% identical to SEQ ID NO: 1. SEQ ID NO: 1 may be found in FIG. 24.

In some embodiments, the bacteria comprise a genome having a sequence that is at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5%, 99.9% or 100% identical to SEQ ID NO: 2. SEQ ID NO: 2 may be found in ASCII format submitted herewith. SEQ NO: 2 is hereby incorporated by reference in its entirety.

In some embodiments, the compositions described herein (e.g., compositions comprising a bacterium comprising nucleotide sequences that are at least partially identical to SEQ ID NO:1 and/or SEQ ID NO:2) further comprise additional bacteria. The additional bacteria may be of the family Lachnospiraceae. In some embodiments, the additional bacteria of the family Lachnospiraceae are of the genus Acetitomaculum, Anaerofilum, Anaerostipes, Blautia, Butyrivibrio, Catenibacterium, Catonella, Clostridium, Coprococcus, Dorea, Epulopiscium, Johnsonella, Lachnobacterium, Lachnospira, Oribacterium, Pseudobutyrivibrio, Roseburia, Ruminococcus, Shuttleworthia or Sporobacterium.

In some embodiments, the bacteria in the methods and compositions provided herein are isolated. As used herein, term “isolated” encompasses a population of bacteria (e.g., a family, species and/or strain of bacteria) that has been (1) separated from at least some of the components with which it was associated when initially produced (whether in nature or in an experimental setting), and/or (2) produced, prepared, purified, and/or manufactured by the hand of man Isolated bacteria may be separated from at least about 10%, about 20%, about 30%, about 40%, about 50%, about 60%, about 70%, about 80%, about 90%, or more of the other components with which they were initially associated. In some embodiments, isolated bacteria are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a population of bacteria that has been separated from at least some of the components with which it was associated either when initially produced or generated (e.g., whether in nature or in an experimental setting), or during any time after its initial production. A bacterial population may be considered purified if it is isolated at or after production, such as from a material or environment containing the bacterial population, and a purified bacterial population may contain other materials up to about 10%, about 20%, about 30%, about 40%, about 50%, about 60%, about 70%, about 80%, about 90%, or above about 90% and still be considered “isolated.” In some embodiments, purified bacterial populations are more than about 80%, about 85%, about 90%, about 91%, about 92%, about 93%, about 94%, about 95%, about 96%, about 97%, about 98%, about 99%, or more than about 99% pure.

In some embodiments, the bacteria that reduce the production of ILC3s do not express a heterologous gene (i.e., are not recombinant). In some embodiments, the bacteria that reduce the production of ILC3s are recombinant. In some embodiments, the bacteria that reduce the production of ILC3s are attenuated. In some embodiments, combinations of species or strains of reduce the production of ILC3s are used in the methods and/or compositions provided herein. In certain embodiments, a combination of at least 2, 3, 4, 5, 6, 7, 8, 9, 10, 11, 12, 13, 14, 15, 16, 17, 18 or 19 of populations of bacteria that reduce the production of ILC3s are used in the methods and/or compositions described herein. In certain embodiments, provided herein is a composition (e.g., a pharmaceutical composition, a dietary supplement or a food product) containing bacteria or combinations of bacteria reduce the production of ILC3s. In some embodiments, the composition further comprises a pharmaceutically acceptable carrier.

In some embodiments, at least 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from among the bacterial populations described herein. 10%, 15%, 20%, 25%, 30%, 35%, 40%, 45%, 50%, 55%, 60%, 65%, 70%, 75%, 80%, 85%, 90%, 95%, 96%, 97%, 98% or 99% of the bacteria in the composition are selected from among the bacterial populations described herein.

In some embodiments, the compositions described herein may include only one population of bacteria described herein or may include two or more populations of the bacteria described herein. For example, 1, 2, 3, 4, 5, 6, 7, 8, 9, 10, 11, 12, 13, 14, 15, 16, 17, 18, 19 or 20 of the populations described herein, in any combination, can be included in the compositions provided herein. In some embodiments, the composition described herein may be a pharmaceutical composition, a dietary supplement, or a food product (e.g., a food or beverage). In some embodiments, the food product is an animal feed.

As described in detail below, the pharmaceutical compositions disclosed herein may be specially formulated for administration in solid or liquid form, including those adapted for oral or rectal administration.

In certain embodiments, the pharmaceutical composition for oral administration described herein comprises an additional component that enables efficient delivery of the bacteria to the colon, in order to more efficiently reduce the number, activity and/or proliferation of colonic ILC3s. In some embodiments, pharmaceutical preparation that enables the delivery of the bacteria to the colon can be used. Examples of such formulations include pH sensitive compositions, such as buffered sachet formulations or enteric polymers that release their contents when the pH becomes alkaline after the enteric polymers pass through the stomach. When a pH sensitive composition is used for formulating the pharmaceutical preparation, the pH sensitive composition can be a polymer whose pH threshold of the decomposition of the composition is between about 6.8 and about 7.5.

Another embodiment of a pharmaceutical composition useful for delivery of the bacteria to the colon is one that ensures the delivery to the colon by delaying the release of the bacteria by approximately 3 to 5 hours, which corresponds to the small intestinal transit time. In some embodiments, the pharmaceutical composition for delayed release includes a hydrogel shell. The hydrogel is hydrated and swells upon contact with gastrointestinal fluid, with the result that the contents are effectively released (released predominantly in the colon). Delayed release dosage units include bacteria-containing compositions having a material which coats or selectively coats the bacteria. Examples of such a selective coating material include in vivo degradable polymers, gradually hydrolyzable polymers, gradually water-soluble polymers, and/or enzyme degradable polymers. A wide variety of coating materials for efficiently delaying the release is available and includes, for example, cellulose-based polymers such as hydroxypropyl cellulose, acrylic acid polymers and copolymers such as methacrylic acid polymers and copolymers, and vinyl polymers and copolymers such as polyvinylpyrrolidone.

Examples of composition enabling the delivery to the colon further include bioadhesive compositions which specifically adhere to the colonic mucosal membrane (for example, a polymer described in the specification of U.S. Pat. No. 6,368,586, hereby incorporated by reference) and compositions into which a protease inhibitor is incorporated for protecting particularly a biopharmaceutical preparation in the gastrointestinal tracts from decomposition due to an activity of a protease.

An example of a system enabling the delivery to the colon is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

Another example of the system enabling the delivery to the colon is a system of delivering a composition to the colon, the system being specifically decomposed by an enzyme (for example, a carbohydrate hydrolase or a carbohydrate reductase) present in the colon. Such a system is not particularly limited, and more specific examples thereof include systems which use food components such as non-starch polysaccharides, amylose, xanthan gum, and azopolymers.

In some embodiments, the compositions described herein further comprise an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments the composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Therapeutic Methods

In certain aspects, provided herein are methods for reduce the amount, activity (e.g., IL-17 production) and/or proliferation of colonic group 3 innate lymphoid cells (ILC3s and/or for treating or preventing a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the methods comprise administering to the subject a bacterium or composition disclosed herein.

The methods described herein can be used to treat any subject in need thereof. As used herein, a “subject in need thereof” includes any subject that has a disease or disorder associated with a pathological immune response (e.g., an inflammatory bowel disease), as well as any subject with an increased likelihood of acquiring a such a disease or disorder.

The bacteria disclosed herein and/or the compositions described herein can be used, for example, as a pharmaceutical composition for preventing or treating (reducing, partially or completely, the adverse effects of) an autoimmune disease. such as chronic inflammatory bowel disease, systemic lupus erythematosus, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a supplement, food, or beverage for improving immune functions; or a reagent for suppressing the proliferation or function of ILC3s.

In some embodiments, the bacteria, compositions and/or methods of the present invention may be used or administered to treat diseases or disorders related to a deleterious immune response, such as asthma, inflammatory disease, skin or organ transplantation, graft-versus-host disease (GVHD), or autoimmune diseases. Examples of autoimmune diseases include, for example, glomerular nephritis, arthritis, dilated cardiomyopathy-like disease, ulceous colitis, Sjogren syndrome, Crohn disease, systemic erythematodes, chronic rheumatoid arthritis, multiple sclerosis, psoriasis, allergic contact dermatitis, polymyosiis, pachyderma, periarteritis nodosa, rheumatic fever, vitiligo vulgaris, insulin dependent diabetes mellitus, Behcet disease, Hashimoto disease, Addison disease, dermatomyositis, myasthenia gravis, Reiter syndrome, Graves' disease, anaemia perniciosa, Goodpasture syndrome, sterility disease, chronic active hepatitis, pemphigus, autoimmune thrombopenic purpura, and autoimmune hemolytic anemia, active chronic hepatitis, Addison's disease, anti-phospholipid syndrome, atopic allergy, autoimmune atrophic gastritis, achlorhydra autoimmune, celiac disease, Cushing's syndrome, dermatomyositis, discoid lupus, erythematosis, Goodpasture's syndrome, Hashimoto's thyroiditis, idiopathic adrenal atrophy, idiopathic thrombocytopenia, insulin-dependent diabetes, Lambert-Eaton syndrome, lupoid hepatitis, some cases of lymphopenia, mixed connective tissue disease, pemphigoid, pemphigus vulgaris, pernicious anema, phacogenic uveitis, polyarteritis nodosa, polyglandular autosyndromes, primary biliary cirrhosis, primary sclerosing cholangitis, Raynaud's syndrome, relapsing polychondritis, Schmidt's syndrome, limited scleroderma (or crest syndrome), sympathetic ophthalmia, systemic lupus erythematosis, Takayasu's arteritis, temporal arteritis, thyrotoxicosis, type b insulin resistance, ulcerative colitis and Wegener's granulomatosis.

In some embodiments, provided herein are bacteria, compositions, and methods for treating an inflammatory bowel disease. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

In some embodiments, administration is in combination with administration of at least one pre-biotic substance (e.g., a prebiotic substance that favors the growth of the bacterial species in the composition over the growth of other human commensal bacterial species). In some embodiments, the prebiotic substance is a nondigestible oligosaccharide. In some embodiments, the prebiotic substance is almond skin, inulin, oligofructose, raffinose, lactulose, pectin, hemicellulose, amylopectin, acetyl-Co A, biotin, beet molasses, yeast extracts, and resistant starch.

In some embodiments, the subject is administered an antibiotic before the composition is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

In some embodiments, the compositions described herein are administered in combination with an immunosuppressive agent. Examples of immunosuppressive agents include corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, anti-TNF inhibitors such as infliximab, adalimumab, certolizumab pegol, golimumab, or etanercept, and combinations thereof. Also described herein is a composition that comprises the bacterial composition and at least one substance selected from the group consisting of corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, anti-TNF inhibitors such as infliximab, adalimumab, certolizumab pegol, golimumab, or etanercept, and combinations thereof.

In some embodiments, the methods provided herein include the step of administering at least one antibiotic before or in combination with, the administration of a composition described herein.

In some embodiments, the methods provided herein include the step of determining the subject's microbiome prior to the administration of a composition described herein. In some embodiments, the selection of the bacteria or combination of bacteria administered to the subject is determined based upon the make-up of the subject's microbiome.

Actual dosage levels of the bacteria in the compositions described herein may be varied so as to obtain an amount of the bacteria which is effective to achieve the desired therapeutic response for a particular patient. A physician or veterinarian having ordinary skill in the art can readily determine and prescribe the effective amount of the composition required.

### Methods of Directed Evolution

In some embodiments, the methods disclosed herein relate to directed evolution and/or methods of identifying novel bacteria species. In some embodiments, the bacteria described herein is man-made or non-naturally occurring bacteria generated through directed evolution. An exemplary method of directed evolution may be found in Example 6. In some embodiments, directed evolution relates to examining gnotobiotic rodents harboring different microbial communities (e.g., a rodent comprising human microbiota). Co-housing of gnotobiotic rodents generates “progeny” that had hybrid microbiotas reflective of both “parents”. Using directed microbial culture techniques, novel species of bacteria may be recovered from hybrid microbiotas. In some embodiments, the bacteria disclosed herein are novel bacterial species, produced by methods of directed evolution disclosed herein. In some aspects, the methods provide herein are used to identify bacteria that correlate with protection or risk of disease by mapping microbe-phenotype relationships in subjects with parental strains and in subjects comprising microbiotas from other species. In some aspects, the methods of directed evolution disclosed herein relate to comparing the microbiomes of disease-resistant or disease-susceptible subjects. In some embodiments, the disease resistant and disease-susceptible subject are co-housed for a period of time, and the change in operation taxonomic units of bacteria in each microbiome are examined.

Provided herein are methods of identifying bacteria that convey disease resistance and/or disease susceptibility by determining a first microbiome profile for a disease-susceptible subject and/or a disease-resistant subject, co-housing the disease-susceptible subject with the disease-resistant subject for a period of time sufficient for the disease-susceptible subject to acquire disease resistance, and, after co-housing, determining a second microbiome profile for the disease-susceptible subject and/or a disease-resistant subject. In some embodiments, the method comprises comparing the first microbiome profile of the disease-susceptible subject with the second microbiome profile of the disease-susceptible subject to identify operational taxonomic units (OTUs) of bacteria that are more prevalent in the second microbiome profile of the disease-susceptible subject than the first microbiome profile of the disease-susceptible subject. Operational taxonomic units (OTUs) of bacteria that are more prevalent in the second microbiome profile of the disease-susceptible subject than the first microbiome profile may confer disease resistance. In some embodiments, the method comprises comparing the first microbiome profile of the disease-susceptible subject with the second microbiome profile to identify operational taxonomic units (OTUs) of bacteria that are less prevalent in the second microbiome profile of the disease-susceptible subject than the first microbiome profile of the disease-susceptible subject. Operational taxonomic units (OTUs) of bacteria that are less prevalent in the second microbiome profile of the disease-susceptible subject than the first microbiome of the disease-susceptible subject profile may confer disease susceptibility.

In some embodiments, the method comprises comparing the first microbiome profile of the disease-resistant subject with the second microbiome profile of the disease-resistant subject to identify operational taxonomic units (OTUs) of bacteria that are more prevalent in the second microbiome profile of the disease-resistant subject than the first microbiome profile of the disease resistant subject. Operational taxonomic units (OTUs) of bacteria that are more prevalent in the second microbiome profile of the disease-resistant subject than the first microbiome profile disease-resistant subject may confer disease susceptibility. In some embodiments, the method comprises comparing the first microbiome profile of the disease-resistant subject with the second microbiome profile of the disease-resistant subject to identify operational taxonomic units (OTUs) of bacteria that are less prevalent in the second microbiome profile of the disease resistant subject than the first microbiome profile of the disease resistant subject. Operational taxonomic units (OTUs) of bacteria that are less prevalent in the second microbiome profile of the disease-resistant subject than the first microbiome of the disease-resistant subject profile may confer disease resistance.

In some aspects, provided herein are methods of identifying bacteria that convey disease susceptibility and/or disease resistance by determining a first microbiome profile for a disease-resistant subject, co-housing the disease-resistant subject with a disease-susceptible subject for a period of time sufficient for the disease-resistant subject to acquire disease susceptibility, and, after co-housing, determining a second microbiome profile for the disease-resistant subject. In some embodiments, the method comprises comparing the first microbiome profile of the disease-resistant subject with the second microbiome profile of the disease-resistant subject to identify operational taxonomic units (OTUs) of bacteria that are more prevalent in the second microbiome profile of the disease-resistant subject than the first microbiome profile of the disease-resistant subject. Operational taxonomic units (OTUs) of bacteria that are more prevalent in the second microbiome profile of the disease-resistant subject than the first microbiome profile of the disease-resistant subject may confer disease susceptibility. In some embodiments, the method comprises comparing the first microbiome profile of the disease-resistant subject with the second microbiome profile of the disease-resistant subject to identify operational taxonomic units (OTUs) of bacteria that are less prevalent in the second microbiome profile of the disease-resistant subject than the first microbiome profile of the disease-resistant subject. Operational taxonomic units (OTUs) of bacteria that are less prevalent in the second microbiome profile of the disease-resistant subject than the first microbiome profile of the disease-resistant subject may confer disease resistance.

In some embodiments, the method comprises comparing the first microbiome profile of the disease-susceptible subject with the second microbiome profile of the disease-susceptible subject to identify operational taxonomic units (OTUs) of bacteria that are more prevalent in the second microbiome profile of the disease-susceptible subject than the first microbiome profile of the disease-susceptible subject. Operational taxonomic units (OTUs) of bacteria that are more prevalent in the second microbiome profile of the disease-susceptible subject than the first microbiome profile of the disease-susceptible subject may confer disease resistance. In some embodiments, the method comprises comparing the first microbiome profile of the disease-susceptible subject with the second microbiome profile of the disease-susceptible subject to identify operational taxonomic units (OTUs) of bacteria that are less prevalent in the second microbiome profile of the disease-susceptible subject than the first microbiome profile of the disease-susceptible subject. Operational taxonomic units (OTUs) of bacteria that are less prevalent in the second microbiome profile of the disease-susceptible subject than the first microbiome of the disease-susceptible subject profile may confer disease susceptibility.

In some embodiments, the disease is inflammatory bowel disease (e.g., Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease), psoriasis, or asthma.

The subject may be a human or a non-human animal, such as a rodent (e.g., mouse or rat). In some embodiments, at least a portion (e.g., at least 10%, at least 20%, at least 30%, at least 40%, at least 50%, at least 60%, at least 70%, at least 80%, at least 90%, or at least 99%) of the subject's microbiome is bacteria from another species. For example, the subject may be a mouse, and have a microbiome comprising bacteria naturally occurring in humans.

The period of time may be any time needed to exchange microbiome bacteria between subject. In some embodiments, the period of time is any time needed to confer disease resistance or disease susceptibility. For example, the time may be 12 hours, 24 hours, 36 hours, 48 hours, 3 days, 5 days, 1 week, 2 weeks, 3 weeks, 6 weeks, or 12 weeks.

### EXEMPLIFICATION

### Example 1

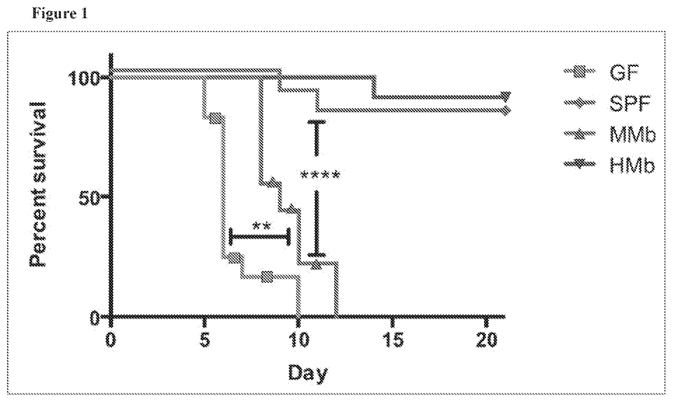

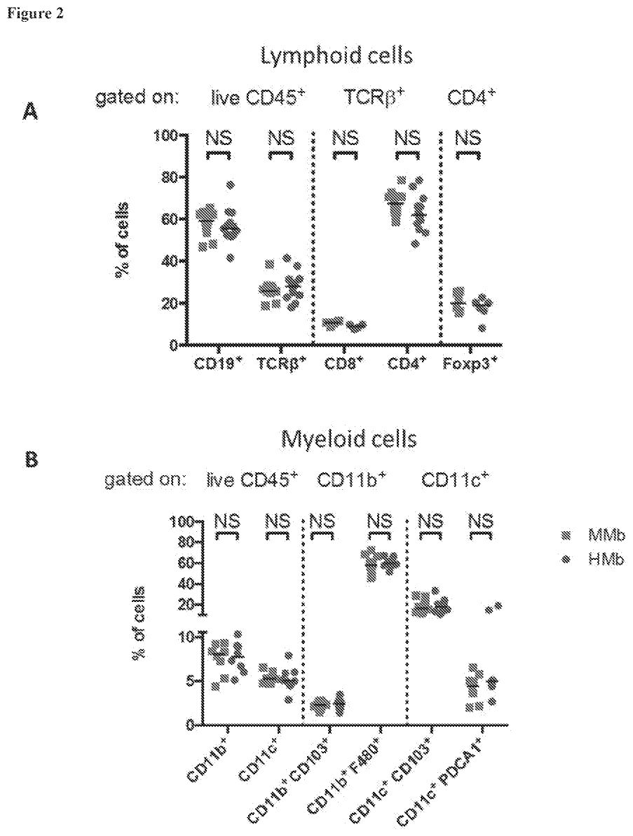

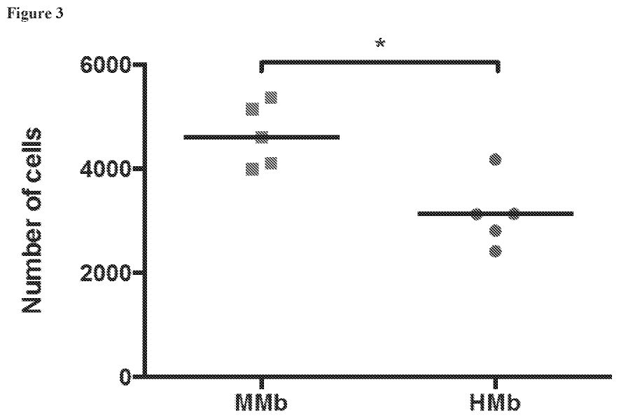

A chemically induced model of colitis was used to explore differences in the colonic immune response of these mice. It was found that, similar to germ-free (GF) mice, MMb mice were sensitive to chemically induced colitis with a 100% mortality rate. In contrast, HMb and SPF mice were resistant to chemically induced colitis (FIG. 1). Flow cytometric analysis of the colonic immune systems revealed that, unlike the small-intestinal immune system, there were no differences between MMb and HMb mice in the numbers of colonic B cells, T cells, or cells of the myeloid lineage (FIG. 2, Panels A and B). However, these mice differed in the number of colonic group 3 innate lymphoid cells (ILC3s), a rare cell type that has been construed as an “innate version” of a T-cell, with higher numbers of ILC3s correlating with more severe disease (FIG. 3). This analysis was done in healthy adult mice (˜8-10 weeks old).

### Example 2

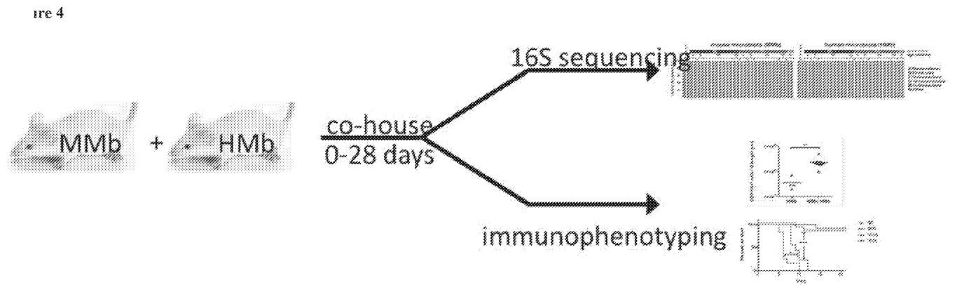

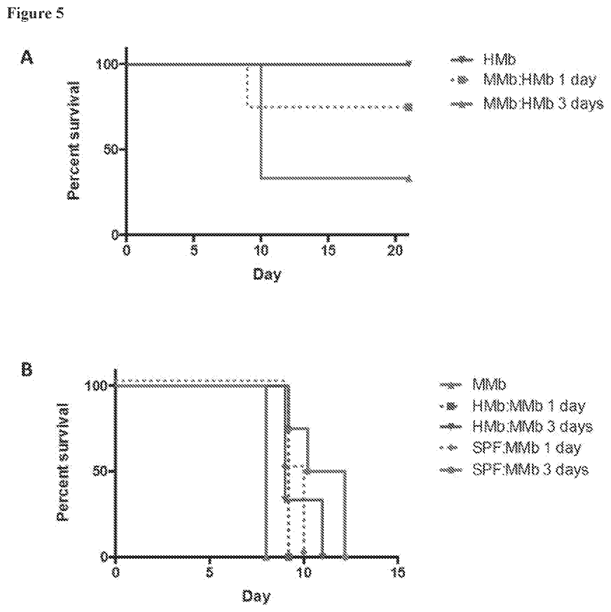

Although an immunological correlate of disease was identified in Example 1, the microbiological cause for these differences was next examined. The general approach was to co-house MMb and HMb mice together (e.g., put the mice in the same cage), taking advantage of the fact that mice are copraphagic to allow for a horizontal transfer of microbes (FIG. 4). If a time period at which there is a difference in the immunological phenotypes can be identified, the microbiota of these co-housed mice can be examined to infer microbes that are associated with disease severity. A near-complete replacement of the HMb microbiota with that of the MMb mouse was observed when HMb mice were co-housed with MMb mice for 3-4 weeks. Accordingly, the next step was to identify the shortest duration of co-housing possible that still results in a difference in the immune phenotypes. Co-housing MMb and HMb mice for 1 day facilitated a bi-directional transmission of microbes that led to an intermediate disease phenotype in both groups (FIG. 5, Panels A and B).

### Example 3

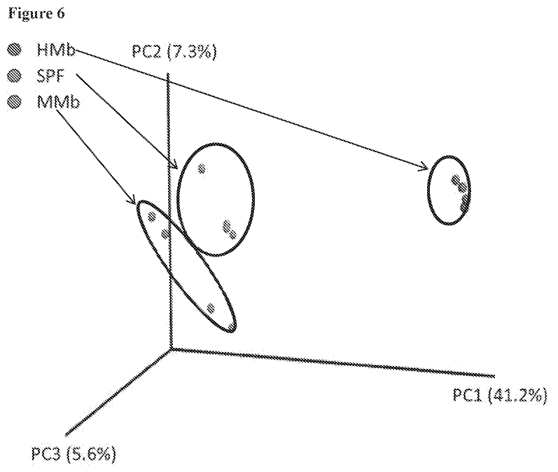

The 16S rDNA from feces of the various mice was sequenced and their microbiotas were analyzed. The data is represented in FIG. 6 in a principal coordinates analysis, which is a graphical representation of differences in beta-diversity. The analysis demonstrates how similar or dissimilar the microbiota is to each other. The closer together 2 dots are in space the more similar is their microbiota.

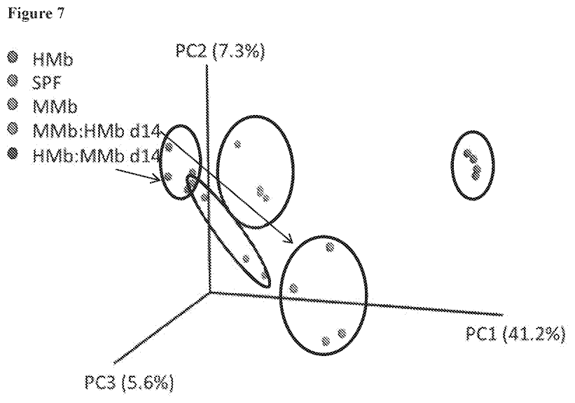

FIG. 7 also includes the data generated from the HMb and MMb mice that were co-housed with each other for 1 day and then singly housed for another 13 days (hence the d14 nomenclature). As the data indicate, co-housing HMb and MMb mice for 1 day results in unique microbial communities.

### Example 4

To identify operational taxonomic units (OTUs; the bioinformatic equivalent of a bacterial species) that are differentially abundant between mouse strains, a free software program called LEfSe was used. 4 different pairwise comparisons were performed. FIG. 8 list the total number of different OTUs present in each pairwise comparison, and the number of differentially abundant taxa (identified by LEfSe) for each comparison. These taxa can be at any taxonomic level (i.e., phyla, order, class, family, genus, species). It was reasoned that if any of these taxa were biologically relevant, they should be shared in each of these comparisons in the same directionality with respect to disease severity. When these criteria were applied, the only taxa that remained was the bacterial family Lachnospiraceae (which contains a number of different genera and species, most of which are poorly characterized if at all).

In sum, bioinformatic analysis of the fecal microbiota of SPF, MMb, HMb, and co-housed mice identified the bacterial family Lachnospiraceae as a microbiological correlate for protection from disease.

### Example 5

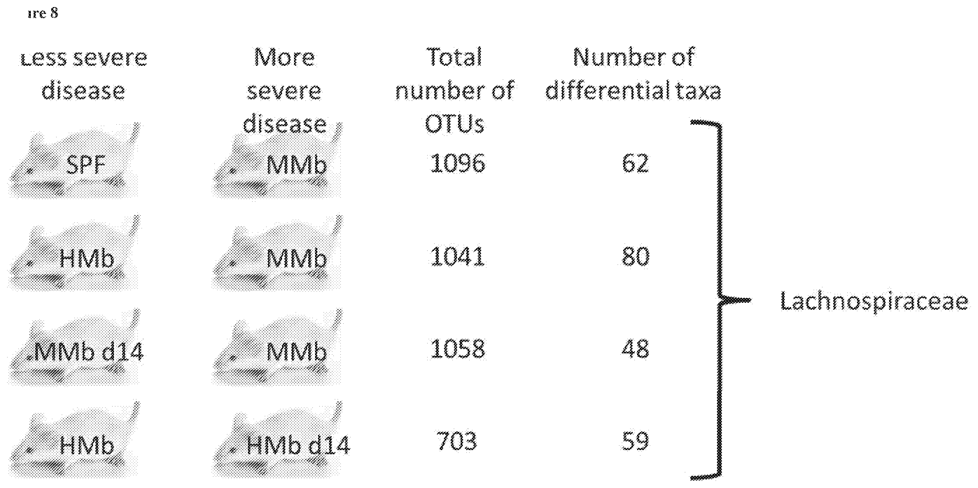

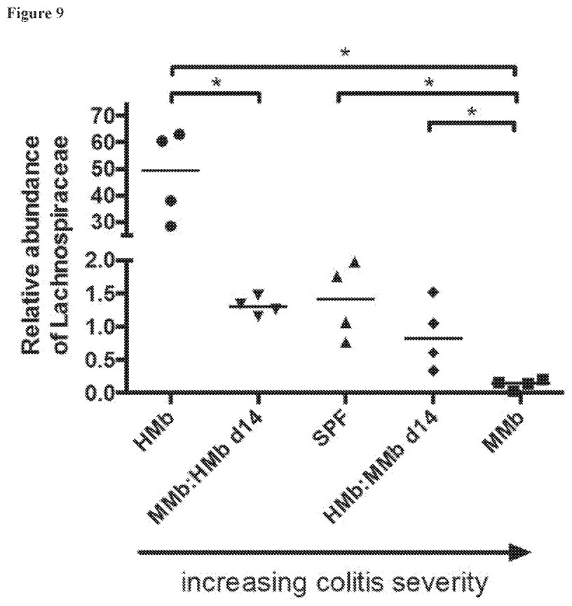

Using the microbiota data for SPF, MMb, HMb, and co-housed mice, the abundance of Lachnospiraceae was next analyzed. It was found that the abundance roughly correlates with disease severity (FIG. 9).

To better define the role of Lachnospiraceae, a semi-selective media was used to culture feces from HMb mice on brain-heart infusion agar plates supplemented with antibiotics (colistin, aztreonam, and gentamicin). The 16S rDNA gene from the starting and final material was then sequenced. As shown in FIG. 10, semi-selective media allowed for a ˜5-fold enrichment of Lachnospiraceae.

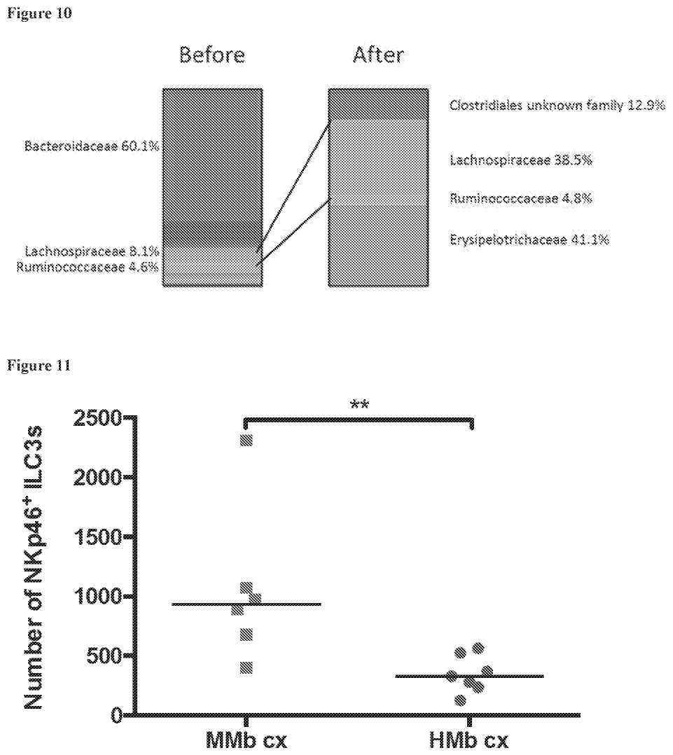

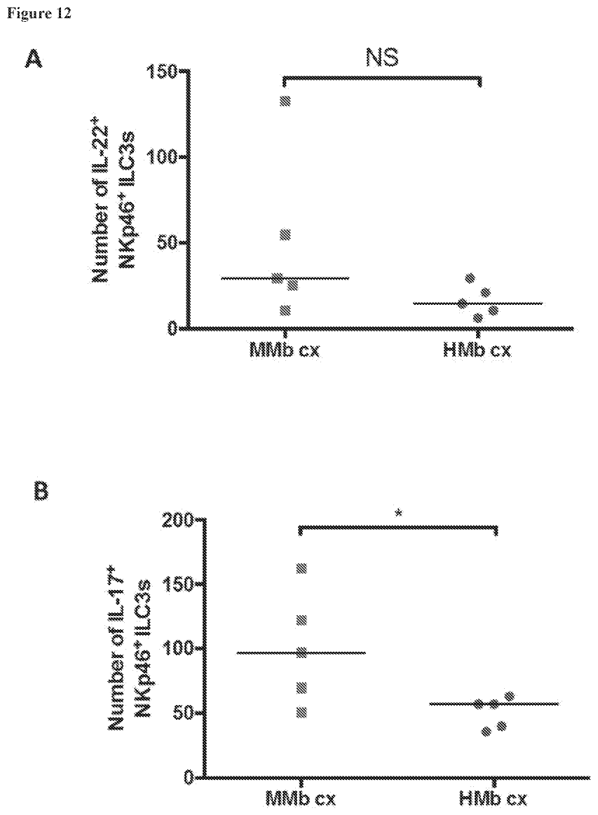

To further define the role of Lachnospiraceae, a semi-selective media was used to culture a Lachnospiraceae-enriched pool of bacteria from HMb mice that—when orally administered to MMb mice—led to decreased numbers of colonic ILC3s. As a control, MMb mice were given bacteria cultured from MMb feces using the same media. MMb mice that received the cultured bacteria from HMb mice had a lower number of colonic group 3 ILCs (similar to HMb mice)(FIG. 11) and protection from colitis-associated mortality, confirming that these organisms are causally linked to ILC3 numbers and disease severity (FIG. 12, Panel A and 13). These group 3 ILCs are known to produce IL-22 and IL-17, two different cytokines relevant to epithelial barrier function and inflammation, respectively. It was found that there was a difference in the IL-17+ group 3 ILCs (FIG. 12, Panel B) with no change in the IL-22+ group 3 ILCs (FIG. 12, Panel A).

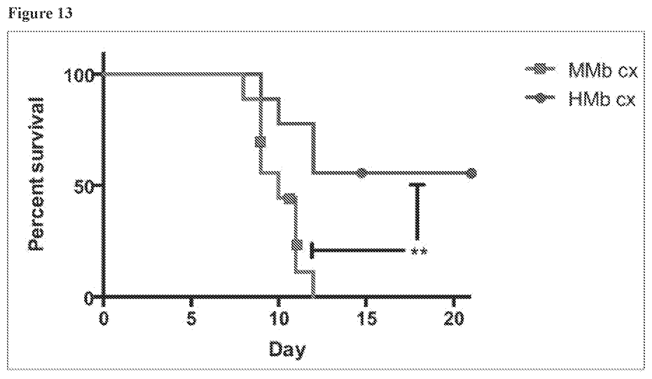

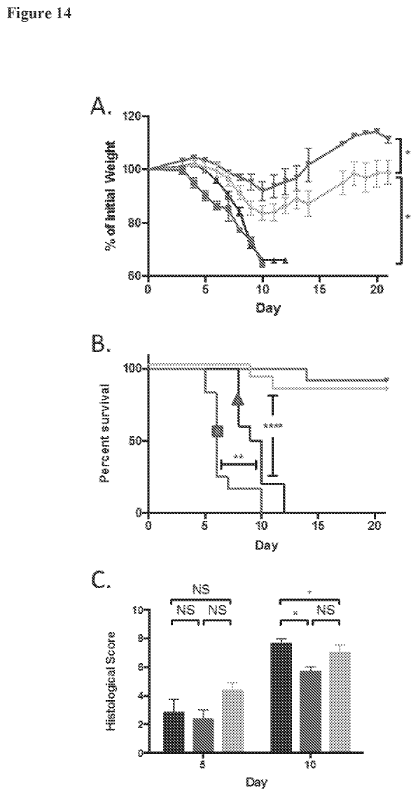

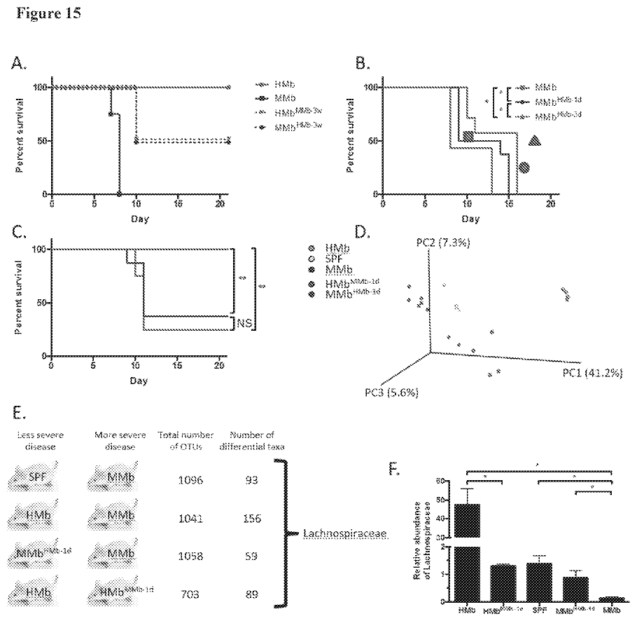

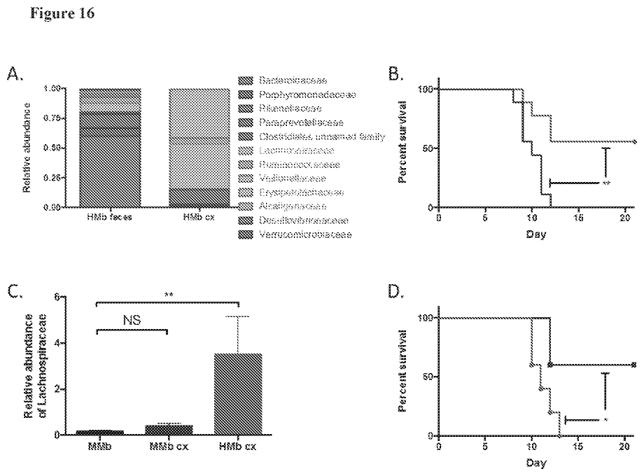

To demonstrate the link between administering Lachnospiraceae and survival from DSS colitis, MMb mice were administered bacteria cultured from HMb feces. These mice were protected from DSS colitis (FIG. 13). The abundance of Lachnospiraceae is increased in these mice, but the abundance of Erysipelotrichaceae (the other main family of bacteria present in the cultured material) was unchanged.

### Example 6

In the present study, it was found that MMb mice are exquisitely sensitive to dextran sodium sulfate (DSS)-induced colitis, exhibiting severe weight loss with a 100% mortality rate (FIGS. 1, Part A and B). In contrast, HMb and specific pathogen-free (SPF) mice lose significantly less weight, with mortality being rare (FIG. 1, Part A and B). This surprising dichotomy in survival between MMb mice and both HMb and SPF mice is not due to a lack of colonic inflammation in HMb or SPF mice: there is no difference in the degree of inflammation at day 5 of disease. However, MMb mice have slightly more severe inflammation than either SPF or HMb mice at day 10 (FIG. 1, Part C).

It was hypothesized that adapting a pedigree analysis to study the microbiome might illuminate a pathway for pinpointing microbes that are more likely to be causally related to disease. Mice were co-housed with different microbiotas to generate “progeny” that had hybrid microbiotas. Assuming that the microbial impact on disease is dominant, mapping of microbe-phenotype relationships in the “parental” strains and in mice with the hybrid microbiotas should enable triangulation of disease-modulating organisms. This idea is analogous to the use of family pedigrees—where the progeny have hybrid genomes reflective of both parents—to identify the genetic basis of disease.

To determine whether the effect of the microbiota is dominant in susceptibility to chemically induced colitis, HMb and MMb mice were co-housed for 3 weeks. Both HMb mice co-housed with MMb mice HMb^{MMb-3w}) and MMb mice co-housed with HMb mice (MMb′⁻³w) had intermediate phenotypes (FIG. 2, Part A), a finding that suggests a bi-directional transfer of microbes. To limit the degree of microbial change, the shortest period of co-housing that still resulted in a phenotypic difference was defined. HMb and MMb mice were co-housed for 1 or 3 days, after which the two groups were separated. On day 14, the mice were challenged with DSS; the rationale for this “rest period” was to allow time for physiological changes to occur. 1 day of co-housing was sufficient to induce significant differences in survival, and the duration of co-housing had a dose-dependent effect (FIG. 2, Part B and C).

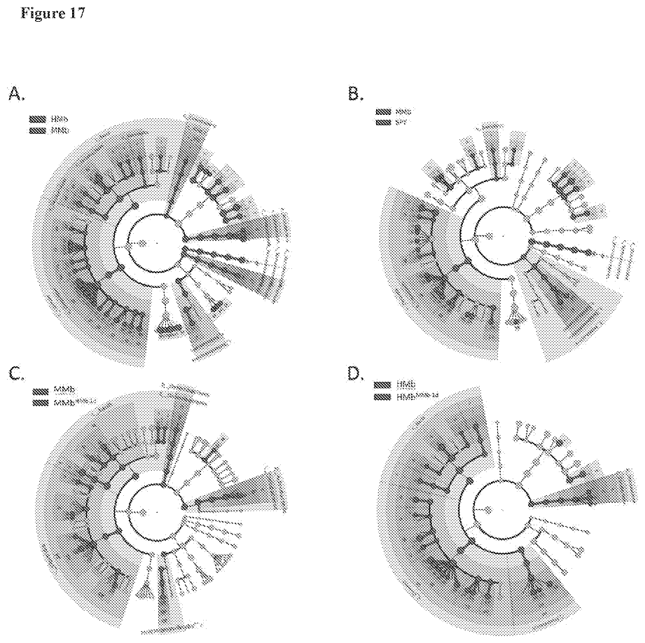

In addition to differences in the severity of DSS-induced disease, it was found that the fecal microbiotas of mice co-housed for 1 day (HMb^{MMb-1d}, MMb^{HMb-1d}) the parental mouse strains (HMb, MMb), and SPF mice were each distinct from one another (FIG. 2, Part D). Bacteria that were differentially abundant in four pairwise comparisons were identified, including pairs where the mice were microbially “related” to one another and where the mice in each pair varied in disease severity. Each pairwise analysis represents a microbiome-wide association study, with each comparison resulting in ˜60-160 differentially abundant bacterial taxa (FIG. 2, Part E, and FIG. 17). If any of these taxa were truly relevant to disease pathogenesis, they would be present in all four of the comparisons. The only taxon that fit this additional criterion was Lachnospiraceae. This family of Gram-positive, anaerobic, non-spore-forming bacteria was associated with survival from DSS-induced colitis. Indeed, the abundance of this family was negligible in MMb mice, intermediate in SPF and co-housed mice, and high in HMb mice (FIG. 2, Part F). Interestingly, the family Lachnospiraceae has been identified in multiple human studies as similarly being inversely correlated with IBD, though the significance of this association remains to be clarified.

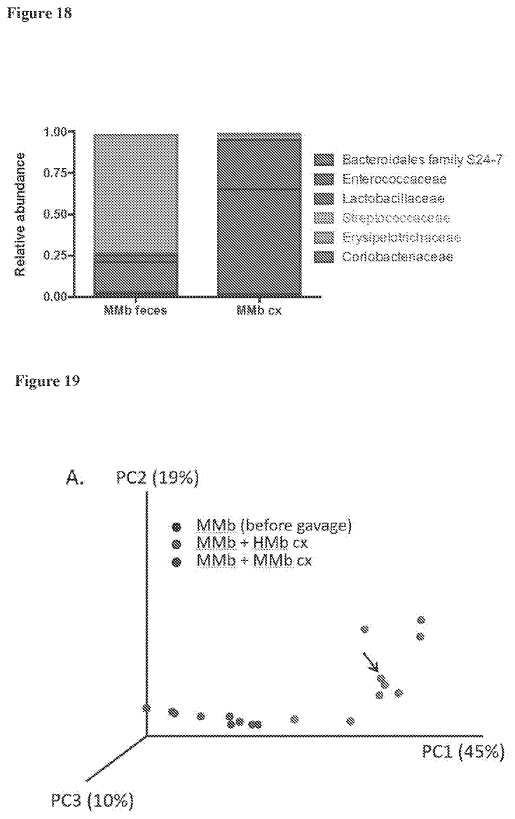

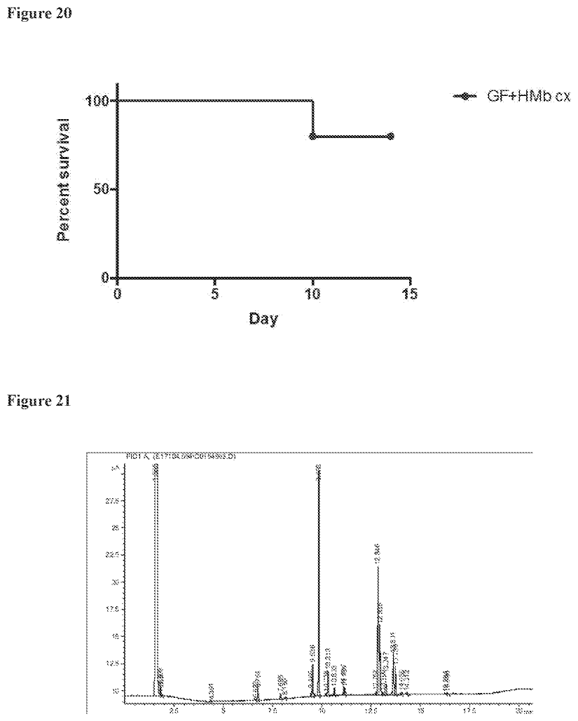

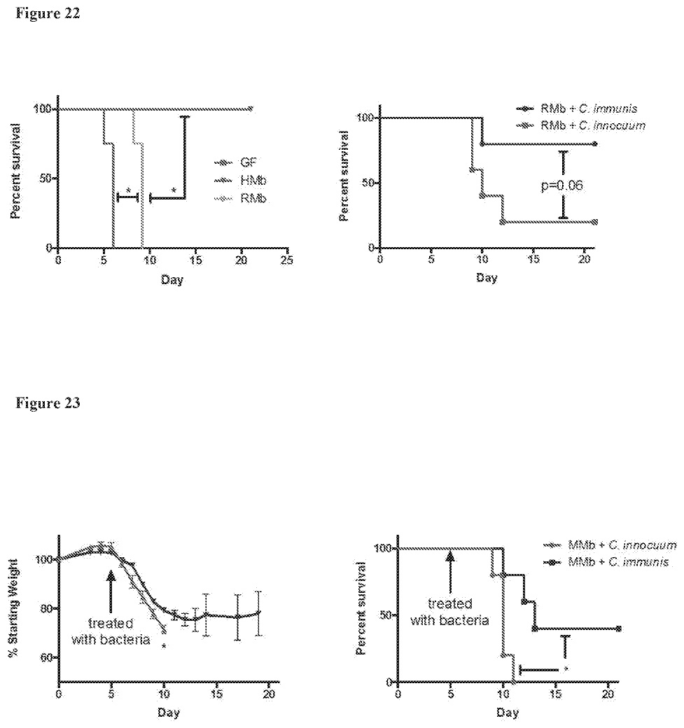

It was then investigated whether Lachnospiraceae is causally linked to improved survival. Feces from HMb mice (HMb cx) were cultured, obtaining a ˜5-fold enrichment on semi-selective medium; the cultured bacterial consortium consisted of ˜40% Lachnospiraceae (FIG. 3, Part A). As a control, feces from MMb mice (MMb cx) were cultured on the same semi-selective medium and found no enrichment of Lachnospiraceae—a finding consistent with the presence of insignificant amounts of this bacterial family in MMb mice (FIG. 18). These bacterial cultures were administered to colitis-prone MMb mice to determine whether Lachnospiraceae can protect against colitis-associated death. Although all MMb mice given MMb cx still succumbed to disease, MMb mice that received HMb cx were remarkably protected (FIG. 3, Part B). The microbiota of these two groups of mice were distinct from each other (FIG. 19); MMb mice that received bacteria cultured from HMb mice had more abundant Lachnospiraceae (FIG. 3, Part C). To ensure that the increased survival was specifically due to the administered bacteria rather than to an unrelated change in the pre-existing MMb microbiota, GF mice were colonized with the HMb cx bacterial mixture and found that these mice were similarly protected from colitis-associated death (FIG. 20).

In order to directly test whether Lachnospiraceae could protect mice against colitis, a single isolate was recovered from HMb feces that was in the family Lachnospiraceae and that accounted for ˜24% of the bacterial abundance in the HMb cx bacterial consortium. The 16S rDNA gene sequence of this isolate shared 98% identity with Clostridium symbiosum but its biochemical properties did not fit either species (see Methods for a more detailed description). Thus, the isolate represents a novel bacterial species, Clostridium immunis. Isolate was administered to MMb mice to determine whether it protected the animals against colitis; as a control, a Clostridium innocuum isolate that accounted for ˜9% of the bacterial abundance in the HMb cx consortium was used. Remarkably, only the MMb mice treated with C. immunis were protected from colitis-associated death (FIG. 3, Part D). Clearly, this isolate is causally related to protection from colitis. This outcome provides mechanistic insight into the clinical observations that the abundance of Lachnospiraceae is inversely correlated to risk of IBD. Moreover, these findings offer critical preclinical support for the use of C. immunis as a probiotic in patients with IBD who have lower abundances of Lachnospiraceae.

The results delineate a bioinformatically straightforward approach that can be used to triangulate specific members of the microbiota that are likely to influence disease pathogenesis. Combination of microbiota analyses from multiple “family pedigrees” allows the researcher to trade sensitivity for increased specificity. A single bacterial taxon was associated with the phenotype of interest, and this correlation was substantiated by directed microbial culture techniques and add-back experiments to fulfill Koch's postulates. Although Koch's postulates and modern revisions of them formally apply only to the identification of pathogens, these findings with a disease-protective commensal organism conform to all of their major tenets. As the field of microbiome research further matures, an increasing number of microbes will most likely be causally linked to protection from disease. Koch's postulates may be expanded to apply to the identification of beneficial organisms, thus ensuring that the burgeoning field of probiotics is subject to the same degree of scientific rigor that has been required in research on microbial pathogenesis.

This “microbial pedigree” analysis may be more generally applicable to human microbiome studies as well. Like the identification of family pedigrees that are informative for genetic analyses, thoughtful selection of study participants and controls may obviate the current trend toward increasingly large, ever more complicated study designs while allowing meaningful comparative analyses. Furthermore, the results from other “-omics” datasets (e.g., transcriptomics, metagenomics, metabolomics) can be analyzed by means of microbial pedigree analysis in order to refine lists of results and determine which are causally related to the phenotype.

### Materials and Methods

### Mice

GF Swiss-Webster (SW) mice were bred and maintained in vinyl isolators in the animal facility at Harvard University. MMb and HMb mice have been bred and maintained at this facility in separate vinyl isolators since their initial characterization. Experimental manipulation of gnotobiotic mice was performed in sterile cages (Innovive; San Diego, Calif.) in which animals received autoclaved food and water. SPF SW mice obtained from Taconic Biosciences were fed an autoclaved diet similar to that given to gnotobiotic mice for 1 week before the start of the study and for the duration of all experiments. Mice used in experiments were age-matched (typically 5-10 weeks old) and drawn randomly from the same litter, where feasible. All procedures were approved by the Harvard Medical Area Standing Committee on Animals and were conducted in accordance with NIH guidelines.

### DSS Colitis

DSS experiments were performed as previously described. In brief, mice were given 4% DSS (molecular weight, 36,000-50,000 Da; MP Biomedicals) ad libitum in their drinking water for 7 days, with the DSS solution changed every 2-3 days. From day 7 until the end of the experiment, the mice were given autoclaved water with no DSS. Animals were weighed every 1-2 days, and any mouse that appeared moribund was sacrificed. A pathologist blinded to treatment groups conducted a histologic assessment of colons. The histologic score represented the combined scores for inflammation and ulceration; both elements were scored 0-4, with 0 being normal.

### Co-Housing Experiments

For co-housing of MMb and HMb mice prior to DSS experiments, two mice per group were placed together in a cage (total, 4 mice) for the indicated period. For a co-housing period of 1 or 3 days, the MMb and HMb mice were separated at the relevant time point, placed into a new sterile cage until day 14 (to allow physiological changes to occur), and then challenged with DSS. For co-housing of MMb and HMb mice for the purpose of fecal microbiota analysis, one mouse per group was placed in a cage (total, 2 mice) for 1 day, after which the mice were separated and individually housed until day 14. Fecal pellets were collected on days 0 and 14 and were frozen at −80° C. until further processed.

16S rDNA Sequencing and Analysis

Fecal samples were added to a tube containing 400 μl of zirconia/silica beads (0.1 mm in diameter; Biospec), 250 μl of 20% sodium dodecyl sulfate, 500 μl of buffer PBI (Qiagen), and 550 μl of phenol-chloroform-isoamyl alcohol (25:24:1; pH 7.9; Ambion) and then homogenized by bead beating for 2.5 min. After centrifugation (10,000×g for 5 min), the DNA in the aqueous phase was purified with a QIAquick PCR purification kit (Qiagen) according to the manufacturer's instructions except that the PE wash step was performed twice. DNA was eluted with 50 μl of EB buffer (Qiagen).

Detailed protocols used for 16S rDNA amplification and sequencing have been described. In brief, the V4 region of the 16S rDNA gene was PCR-amplified (35 cycles, primers 515F and 806R) in triplicate; 1 μl of purified DNA and 5′ Hot Master Mix (Five Prime) were used Amplicons were quantified by Quant-It (Invitrogen), pooled in equimolar concentration, and size-selected (375-425 bp) on the Pippen Prep (Sage Sciences; Beverly, Mass.) to reduce nonspecific amplification products. Sequencing was performed on a MiSeq sequencer (Illumina; 2×250 bp paired-end reads using V2 chemistry).

Microbial diversity was analyzed with Quantitative Insights into Microbial Ecology (QIIME versions 1.8 and 1.9). The closed-reference operational taxonomic unit (OTU) workflow in QIIME and the Greengenes reference database (May 2013) were used to cluster reads into OTUs with 97% identity and to assign taxonomy to representative OTUs. OTU tables were rarified to a depth of 40,000 and 13,000 sequences per sample for experiments described in FIGS. 2 and 3, respectively. Principal-coordinates beta-diversity visualizations were created with Emperor as packaged in QIIME. The Linear Discriminant Analysis Effect Size (LEfSe) Galaxy module (http://huttenhower.sph.harvard.edu/galaxy/) was used for additional statistical analyses.

### Bacterial Culture

Serial dilutions of feces from MMb and HMb mice were spread on brain—heart infusion (BHI) agar supplemented with colistin (10 μg/ml), gentamicin (6 μg/ml), and aztreonam (5 μg/ml), and cultures were incubated for 7 days in an anaerobic chamber (Coy Industries; Grass Lake, Mich.). MMb cx and HMb cx samples were harvested from plates that contained ˜500-1000 colonies, resuspended in pre-reduced BHI agar, and frozen at −80° C. until needed. To recover specific isolates, HMb feces were cultured on the medium described above for 5-14 days and picked ˜60 individual colonies. These colonies were inoculated into pre-reduced chopped meat medium with glucose (Anaerobe Systems; Morgan Hill, Calif.), and genomic DNA was isolated with the DNeasy Blood and Tissue Kit (Qiagen). We compared the 16S rDNA gene sequence from each of these isolates to Genbank and the Greengenes reference database to identify bacterial taxonomy.

### Probiotic Administration to Mice

MMb and GF mice received orally administered MMb cx, HMb cx, Clostridium innocuum, or Clostridium immunis (100 μl; ˜10⁸-10⁹ colony-forming units). Seven days later, the mice were challenged with DSS. In some experiments, fecal samples were collected before and 7 days after probiotic administration for microbiota analysis.

Characterization of C. immunis

The Lachnospiraceae isolate disclosed herein stains gram-negative, is resistant to colistin differential disks (Anaerobe Systems), and is sensitive to vancomycin and kanamycin differential disks (Anaerobe Systems). Taken together, these findings are consistent with the isolate's being a gram-positive organism. The closest match for the 16S rDNA gene sequence of this isolate is Clostridium symbiosum, with which it shares 98% identity; MALDI-TOF analysis by VITEK MS (BioMerieux; Durham, N.C.) revealed that the closest match for this isolate is Clostridium clostridioforme; and MALDI-TOF analysis with the MALDI Biotyper (Bruker; Billerica, Mass.) was unable to provide a species- or genus-level identification. Biochemical testing demonstrated that the isolate—unlike C. symbiosum—is resistant to 20% bile and does not produce acid from mannose. Moreover, the isolate—unlike C. clostridioforme—produces abundant amounts of butyrate from peptone-yeast extract-Fildes solution-glucose broth. Given these genetic, proteomic, and biochemical differences from its closest relatives, this isolate represents the type strain of Clostridium immunis, a novel species within the family Lachnospiraceae. Below is a preliminary annotation of the Clostridium immunis genome and a summary of biochemical properties of Clostridium immunis.

TABLE 1
Annotation of Clostridium immunis genome.

| Genome Feature | C. immunis |
|---|---|
| Size | 5.44 Mb |
| % GC | 47.7 |
| N50 | 3,429,461 |
| Number of contigs | 7 |
| Number of coding sequences | 5905 |
| Number of RNAs | 75 |
| Virulence genes | None detected; predicted |
| | to be avirulent |
| Antimicrobial Resistance | tetracycline |
| Genes | |

TABLE 2
Biochemical Properties of C. immunis vs
Representative Bacterial Stain

| | C. | C. | C. |
|---|---|---|---|
| | immunis | symbiosum | clostridioforme |
| SCFA production | Acetate, | Acetate, | acetate |
| from PYG-F | butyrate | butyrate, | |
| | (lactate | lactate | |
| | not tested) | | |
| Ferments | No | add | Weak acid |
| mannose | | | |
| Growth in 20% | Yes | no | Yes |
| bile | | | |
| Esculin hydrolysis | No | No | Yes |

### Statistics

Sample size estimates for each experiment were based on prior lab experience. Prism 6 (GraphPad Software; La Jolla, Calif.) was used for all statistical analyses. All p values were calculated by unpaired, two-tailed Student's t or Mann-Whitney test, as appropriate. For survival curves, p values were calculated by the Mantel-Cox test.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of reducing the amount, activity and/or proliferation of colonic group 3 innate lymphoid cells (ILC3s) in a subject comprising administering to the subject a composition comprising a bacteria of the family Lachnospiraceae.

2. The method of claim 1, wherein the bacteria of the family Lachnospiraceae are of the genus Acetitomaculum, Anaerofilum, Anaerostipes, Blautia, Butyrivibrio, Catenibacterium, Catonella, Clostridium, Coprococcus, Dorea, Epulopiscium, Johnsonella, Lachnobacterium, Lachnospira, Oribacterium, Pseudobutyrivibrio, Roseburia, Ruminococcus, Shuttleworthia or Sporobacterium.

3. The method of claim 1, wherein the subject suffers from or is predisposed to an inflammatory bowel disease, psoriasis, or asthma.

4. The method of claim 1, wherein the inflammatory bowel disease is Crohn's disease, ulcerative colitis, irritable bowel syndrome, microscopic colitis, lymphocytic-plasmocytic enteritis, coeliac disease, collagenous colitis, lymphocytic colitis and eosinophilic enterocolitis, indeterminate colitis, infectious colitis, pseudomembranous colitis, ischemic inflammatory bowel disease or Behcet's disease.

5. (canceled)

6. The method of claim 1, wherein at least 10% at least 25%, at least 50%, at least 75%, at least 90%, or at least 99% of the bacteria in the composition are of the family Lachnospiraceae.

7-12. (canceled)

13. The method of claim 1, wherein the composition is a food product supplemented with the bacteria.

14. The method of claim 1, wherein the composition is administered orally.

15. The method of claim 1, wherein the composition is administered rectally.

16. The method of claim 1, wherein the bacteria are live, replication competent bacteria.

17. The method of claim 1, wherein the administration of the composition reduces the number of IL-17 expressing colonic ILC3s in the subject.

18. The method of claim 1, further comprising administering a pre-biotic to the subject.

19. The method of claim 1, further comprising administering an antibiotic to the subject prior to the administration of the composition.

20. A pharmaceutical composition comprising a bacteria of the family Lachnospiraceae and a pharmaceutically acceptable carrier.

21. The composition of claim 20, wherein the bacteria of the family Lachnospiraceae are of the genus Acetitomaculum, Anaerofilum, Anaerostipes, Blautia, Butyrivibrio, Catenibacterium, Catonella, Clostridium, Coprococcus, Dorea, Epulopiscium, Johnsonella, Lachnobacterium, Lachnospira, Oribacterium, Pseudobutyrivibrio, Roseburia, Ruminococcus, Shuttleworthia or Sporobacterium.

22. The composition of claim 20, wherein at least 10%, at least 25%, at least 50%, at least 75%, at least 90%, or at least 99% of the bacteria in the composition are of the family Lachnospiraceae.

23-27. (canceled)

28. The composition of claim 20, wherein the composition is a food product supplemented with the bacteria.

29. The composition of claim 20, wherein the composition is formulated for oral administration.

30. The composition of claim 20, wherein the composition is formulated for rectal administration.

31. The composition of claim 20, wherein the bacteria are live, replication competent bacteria.

32. The composition of claim 20, further comprising a pre-biotic.
